(19) United States
(12) Patent Application Publication
(10) Pub. No.: US 2023/0303645 A1
(43) Pub. Date: Sep. 28, 2023

(54) COMPOSITIONS AND METHODS FOR ENHANCING NUCLEIC ACID THERAPEUTICS

(71) Applicant: The Trustees of Columbia University in the City of New York, New York, NY (US)

(72) Inventors: Fatemeh Momen HERAVI, Teaneck, NJ (US); Akiva MINTZ, Paramus, NJ (US)

(21) Appl. No.: 18/312,859

(22) Filed: May 5, 2023

Related U.S. Application Data
(63) Continuation of application No. PCT/US21/58237, filed on Nov. 5, 2021.
(60) Provisional application No. 63/110,702, filed on Nov. 6, 2020.

(51) Int. Cl. C07K 14/47; C12N 15/113; C12N 15/86

(52) U.S. Cl. CPC C07K 14/4703; C12N 15/113; C12N 15/86

(57) ABSTRACT

The compositions for enhancing nucleic acid therapeutics are used in the treatment or prevention of diseases or conditions. The compositions improve the use of nucleic acid therapeutics with the use of enhancing or stabilizing elements, such as RNA-induced silencing complex (RISC) proteins. The compositions include at least one nucleic acid therapeutic or a polynucleotide encoding at least one nucleic acid therapeutic; at least one enhancing or stabilizing element, mutant, variant or modified form thereof, or a polynucleotide encoding at least one enhancing or stabilizing element, mutant, variant or Modified form thereof; and a delivery vehicle, where the delivery vehicle may be exosomes, microvesicles, apoptotic bodies, oncosomes, microparticles, extracellular vesicles, liposomes, nanoparticles, plasmids or vectors. The exosomes or extracellular vesicles may be engineered to be substantially devoid of endogenous nucleic acids by downregulating or inhibiting at least one protein involved in sorting or loading nucleic acids into exosomes or extracellular vesicles.

### CROSS-REFERENCE TO RELATED APPLICATION

This application is a continuation of International Application No. PCT/US2021/058237, filed on Nov. 5, 2021, titled “COMPOSITIONS AND METHODS FOR ENHANCING NUCLEIC ACID THERAPEUTICS,” which claims the benefit of U.S. Provisional Patent application No. 63/110,702, titled “COMPOSITIONS AND METHODS FOR ENHANCING NUCLEIC ACID THERAPEUTICS,” filed on Nov. 6, 2020, the entirety of the disclosures of which are hereby incorporated herein by this reference.

### TECHNICAL FIELD

The disclosure of the present patent application relates to nucleic acid therapeutics, and particularly to compositions for improving the use of nucleic acid therapeutics with the use of enhancing or stabilizing elements, such as RNA-induced silencing complex (RISC) proteins.

### BACKGROUND ART

Gene silencing by RNA interference (RNAi) is a therapeutic approach for the treatment of various diseases. There is an emerging interest in therapeutic small RNA (less than 200 nucleotides in length), as the U.S. Food and Drug Administration (FDA) recently approved the first small-interfering RNA (siRNA). MicroRNAs (miRs) are endogenous, small non-coding RNAs (18-22 nucleotides) that regulate genes post-transcriptionally by interacting with the 3′-untranslated (3′UT) region of mRNA and influence the gene regulation of essential biological pathways. Abundant evidence has shown that dysregulation of miRs plays a mechanistic role in initiation and progression of various diseases and conditions, including cancer, autoimmune diseases, and metabolic diseases. These changes have been attributed to genomic alterations of tumors or germline variants (amplifications, deletion, or chromosomal changes), abnormal transcriptional control of miRs, dysregulated epigenetic changes and alterations in the miR biogenesis machinery.

In cancer, miRs may function as either tumor suppressors or oncogenes by virtue of miR-specific and context-dependent mechanisms. The dysregulated miRs have been shown to effect hallmarks of cancer, including induction of epithelial mesenchymal transition, sustained proliferation, evasion of anti-tumor mechanisms, promotion of metastases, evading of apoptosis, and induction of angiogenesis. miRs play a role in pathogenesis of metabolic diseases, such as Non-Alcoholic SteatoHepatitis (NASH) and diabetes.

Additionally, miRs act as master regulators of gene expression by targeting multiple genes and pathways simultaneously. This unique ability of miRs makes miRs very suitable therapeutic candidates. Although the emergence of miR-based therapeutics has not yet translated into FDA-approved candidates for medical intervention, several candidate therapeutics are in clinical development or in phase 1 or phase 2 clinical trials for different conditions, including miR-122 (Miravirsen) for treatment of hepatitis C, MRG 110 (a locked nucleic acid (LNA)-modified antisense oligonucleotide to inhibit the function of miR-92) for treatment of heart failure, miR-29 (MRG-201) to treat keloid and scar tissue formation, and miR-155 (Cobomarsen; MRG-106) to treat T-cell lymphoma.

The therapeutic use of miRs, inhibitors or similar nucleic acid therapeutics is not restricted to cancer. For example, it has been shown that miR-132 is a significant factor in liver fibrosis and that liver fibrosis can be attenuated by delivering LNA-anti-miR-132 inhibitors (FIGS. 3A-3E). The delivery of miR inhibitors or miR mimics or other nucleic acid for therapeutics to target cells has been a very active area of research in the past few years.

For FIGS. 3A-3E, LNA-anti-miRNA-132 was delivered to mice. FIG. 3A shows the division of sample mice used in the LNA-anti-miRNA-132 delivery, where C57BL/6 male mice (n=8) were injected either with LNA-scrambled control, LNA-anti miR-132 (@ 15 mg/kg) or saline, intraperitoneal as shown. Additionally, some mice received either corn oil or CC14 (i.p.: 0.6 ml/kg of body weight) for indicated times. FIG. 3B is a graph comparing the results for corn oil and CC14, where RNA isolated from the liver was used to determine miR-132 expression by real-time qPCR using a Taqman™ microRNA assay. FIG. 3C is a graph comparing the results for corn oil and CC14, where RNA isolated from the liver was used to determine miR-212 expression by real-time qPCR using a Taqman™ microRNA assay. In FIGS. 3B and 3C, * indicates p<0.05 compared to oil and saline treated mice, and # indicates p<0.05 compared to LNA-scrambled control treated mice after CC14 treatment. Data represent mean+standard error of mean (SEM). The Mann-Whitney test was employed for statistical analysis. FIG. 3D compares images showing sirius red staining of paraffin embedded liver sections. FIG. 3E shows a western blot image, where 20 μg of whole liver lysate protein was used to determine a smooth muscle actin expression. Beta actin was used as a loading control.

Additionally, FIG. 1 is a graph illustrating the differential expression of tumor suppressor miRs in oral squamous cell carcinoma (OSCC) and adjacent tissue. miRs were quantified in the OSCC tissue and adjacent tumor tissue using quantitative reverse transcription PCR (RT-qPCR). The data was normalized to RNU48. (n=28). In FIG. 1, * indicates p<0.05. FIG. 2A is a graph comparing cell proliferation for miR-34a introduced to CAL27 cells (an oral cancer cell line) via electroporation against a control. Cell proliferation was quantified by MTT assay. FIG. 2B shows a representative flow cytometry plot of annexin V (FITC) vs. propidium iodide in control miR treated cells or miR-34a-5p treated OSCC cells (25 nM, 48 hours). Early apoptotic cells were defined as propidium iodide low and Annexin-V high. The percentage of early apoptotic cells was determined by early apoptosis detection assay in two different head and neck cancer cell lines (HTB-43 and CAL27 cells). In FIG. 2A, the * indicates p<0.05.

siRNA is another class of nucleic acid-based drugs able to prevent gene expression by interaction with mRNA before its translation. Like miRs, siRNAs have phenomenal potential to treat human diseases due to their ability to silence the expression of disease-causing genes. miRs, siRNAs and other RNAs in the naked forms are typically degraded by RNases, such RNase A-type nucleases in the blood, and LNA miRs induce immunogenicity. Viral delivery methods for the inhibition of miRs through the expression of transcripts complementary to mature miR sequences or ectopic expression of siRNAs have been investigated. However, their clinical use has been limited due to the immunogenicity, off-target effects of the viruses, lack of targeting moieties, and incorporation into the genome. Non-viral synthetic vectors, such as liposomes and nanoparticles, are another class of delivery vehicles that can be used to deliver nucleic acid cargos, including miR-based therapy and siRNAs. While they naturally lack targeted cell-specific delivery, targeting moieties such as peptides or antibodies can be conjugated to them. Some challenges facing synthetic delivery vectors as therapeutic vehicles include biocompatibility, toxicity, immunogenic potential, problems with therapeutic cargo release, and non-specific uptake by macrophages.

One of the challenges in using RNAi is limited half-life of some of the RNA molecules and their delivery to specific cells. Thus, there is an unmet need for improvement for the therapeutic use of nucleic acid therapeutics, including microRNA-based therapies and siRNAs. Thus, compositions and methods for enhancing nucleic acid therapeutics solving the aforementioned problems is desired.

### DISCLOSURE

The compositions for enhancing nucleic acid therapeutics are used in the treatment or prevention of diseases or conditions, such as, for example, cancer or diseases or disorders of the liver. The compositions improve the use of nucleic acid therapeutics with the use of enhancing or stabilizing elements, such as RNA-induced silencing complex (RISC) proteins. The compositions include at least one nucleic acid therapeutic or a polynucleotide encoding at least one nucleic acid therapeutic; at least one enhancing or stabilizing element, mutant, variant or modified form thereof, or a polynucleotide encoding at least one enhancing or stabilizing element, mutant, variant or modified form thereof; and a delivery vehicle, where the delivery vehicle may be exosomes, microvesicles, apoptotic bodies, oncosomes, microparticles, extracellular vesicles, liposomes, nanoparticles, plasmids or vectors. When exosomes or extracellular vesicles are used as the delivery vehicle, the exosomes or the extracellular vesicles may optionally be engineered to be substantially devoid of endogenous nucleic acids by downregulating or inhibiting at least one protein involved in sorting or loading nucleic acids into exosomes or extracellular vesicles. The exosome or the extracellular vesicle may optionally include at least one targeting moiety or therapeutic molecule expressed on a surface of the exosome or the extracellular vesicle. The making of engineered exosomes or extracellular vesicles substantially devoid of endogenous nucleic acids is described in PCT application no. WO 2021/041473 A1, which is hereby incorporated by reference in its entirety. The at least one enhancing or stabilizing element, mutant, variant or modified form thereof, or the polynucleotide encoding at least one enhancing or stabilizing element, mutant, variant or modified form thereof, may be ectopically expressed or overexpressed in the delivery vehicle. As used herein, the term “ectopically expressed” refers to abnormal gene expression, such that the ectopic expression in the delivery vehicle by the at least one enhancing or stabilizing element, mutant, variant or modified form thereof, or the polynucleotide encoding at least one enhancing or stabilizing element, mutant, variant or modified form thereof, is expressed abnormally in the delivery vehicle; i.e., the delivery vehicle, in its normal or unmodified state, would not exhibit this expression. As a non-limiting example, such ectopic expression or overexpression may occur through artificial manipulation of the delivery vehicle.

As used herein, the terms “may” and “may optionally” refer to optional embodiments.

As a non-limiting example, the vector may be a viral vector. Non-limiting examples of the nucleic acid therapeutic include RNA, at least one small interfering RNA (siRNA), at least one small hairpin RNA (shRNA), at least one microRNA (miRNA), a double-stranded RNA (dsRNA), an antisense nucleic acid, a locked nucleic acid (LNA), a microRNA inhibitor, a chemically-modified microRNA, a chemically-modified siRNA, a chemically-modified shRNA, a chemically-modified dsRNA, a chemically-modified antisense nucleic acid, or a chemically-modified microRNA inhibitor.

Non-limiting examples of the enhancing or stabilizing element include a portion of an RNA-induced silencing complex (RISC) protein, a whole RISC, a mutant, variant or modified form thereof, or any combination of RISC complex. The RISC protein may be Argonaute 2, a mutant, variant or modified form thereof. As another non-limiting example, the enhancing or stabilizing element may be a ribonucleoprotein. The ribonucleoprotein may be GW182, DCP1/CDP2, PUM1, HUR, or mutants, variants or modified forms thereof.

These and other features of the present subject matter will become readily apparent upon further review of the following specification.

### BRIEF DESCRIPTION OF THE DRAWINGS

FIG. 1 is a graph illustrating the differential expression of tumor suppressor miRs in oral squamous cell carcinoma (OSCC) and adjacent tissue. miRs were quantified in the OSCC tissue and adjacent tumor tissue using quantitative reverse transcription PCR (RT-qPCR).

FIG. 2A is a graph comparing cell proliferation for miR-34a introduced to CAL27 cells (an oral cancer cell line) via electroporation against a control. Cell proliferation was quantified by MTT assay.

FIG. 2B shows a representative flow cytometry plot of annexin V (FITC) vs. propidium iodide in control miR treated cells or miR-34a-5p treated OSCC cells (25 nM, 48 hours).

FIG. 3A illustrates the division of sample mice used in LNA-anti-miRNA-132 delivery testing, where C57BL/6 male mice (n=8) were injected either with LNA-scrambled control, LNA-anti miR-132 (@ 15 mg/kg) or saline, intraperitoneal as shown. Additionally, some mice received either corn oil or CC14 (i.p.: 0.6 ml/kg of body weight) for indicated times.

FIG. 3B is a graph comparing the results for corn oil and CC14, where RNA isolated from the liver was used to determine miR-132 expression by real-time qPCR using a Taqman™ microRNA assay.

FIG. 3C is a graph comparing the results for corn oil and CC14, where RNA isolated from the liver was used to determine miR-212 expression by real-time qPCR using a Taqman™ microRNA assay.

FIG. 3D compares images showing sirius red staining of paraffin embedded liver sections.

FIG. 3E shows a western blot image, where 20 μg of whole liver lysate protein was used to determine a smooth muscle actin expression. Beta actin was used as a loading control.

FIG. 4 is a graph illustrating the silencing of AGO2 in an oral cancer cell line, both with or without copGFP plasmid or AGO2 overexpression plasmid. Actinomycin D was administered, and levels of miR-34a were determined by RT-qPCR.

FIG. 5A is a graph comparing CAL27 cells treated with Alix siRNA against a control, where the media was changed after 16 hours and the exosomes were isolated after 48 hours.

FIG. 5B is a graph comparing CAL27 cells treated with standard CAL27 exosomes and engineered exosomes derived from CAL27.

FIG. 5C is a graph comparing engineered exosomes loaded with Ce139 against controls, where the level of the miR was quantified in the recipient cells by RT-qPCR (n=4 per group).

FIG. 6 shows fluorescent cell images for KRAS 4B wild type cells treated with PKH26 (red fluorescent)-labeled safeEXO containing ATP or no ATP as indicated. SafeEXO is a platform that manipulates the exosome creation machinery in producer cells to significantly minimize endogenous RNA loading into exosomes.

FIG. 7 compares western blots for exosomes isolated from HEK293T cells transfected with an Ago2 expressing plasmid against a control exosome, a control lysate, and an Ago2 lysate.

FIG. 8 is a graph comparing relative expression of miR-139-5p of control exosomes, Ago2 expressing exosomes, control exosomes loaded with miR-139-5p, and Ago2 expressing exosomes loaded with miR-139-5p, each co-cultured with HEK293T cells.

FIG. 9 is a graph comparing relative expression of Zeb1 after 48 hours in the samples of FIG. 8. Zeb 1 is a direct target of miR-139-5p.

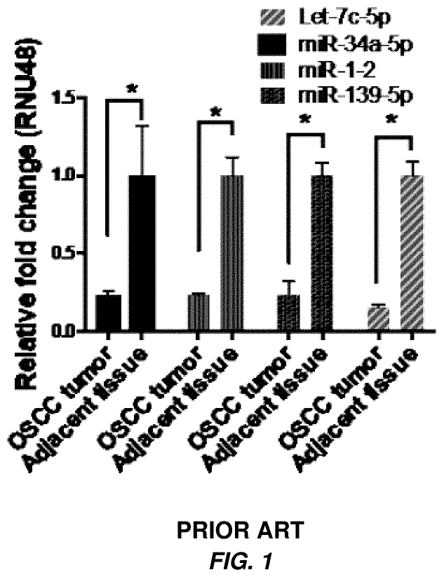

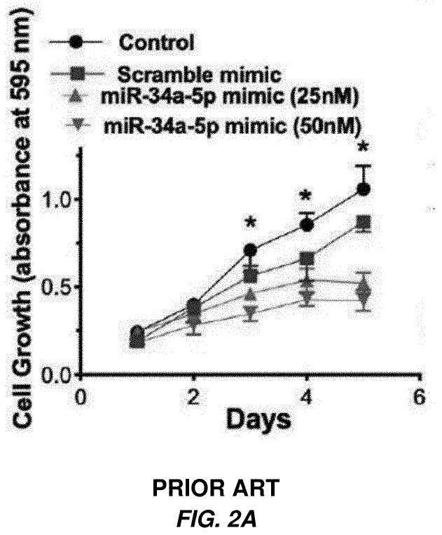

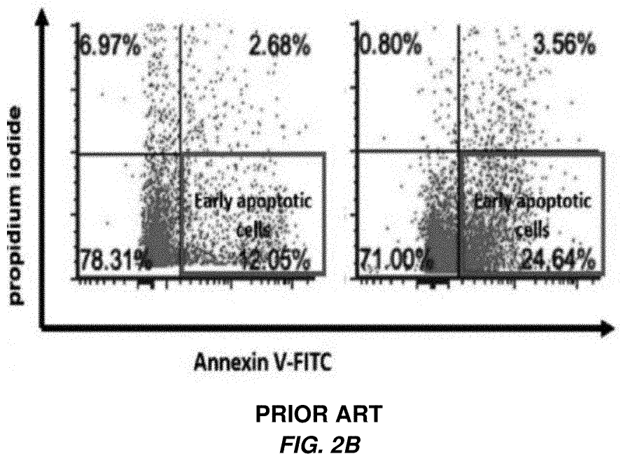

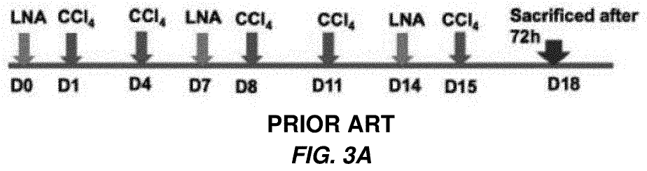

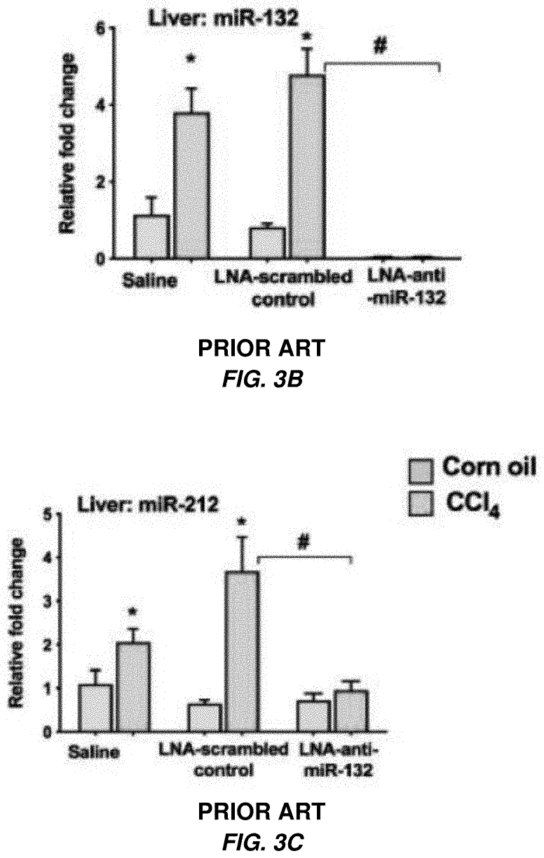

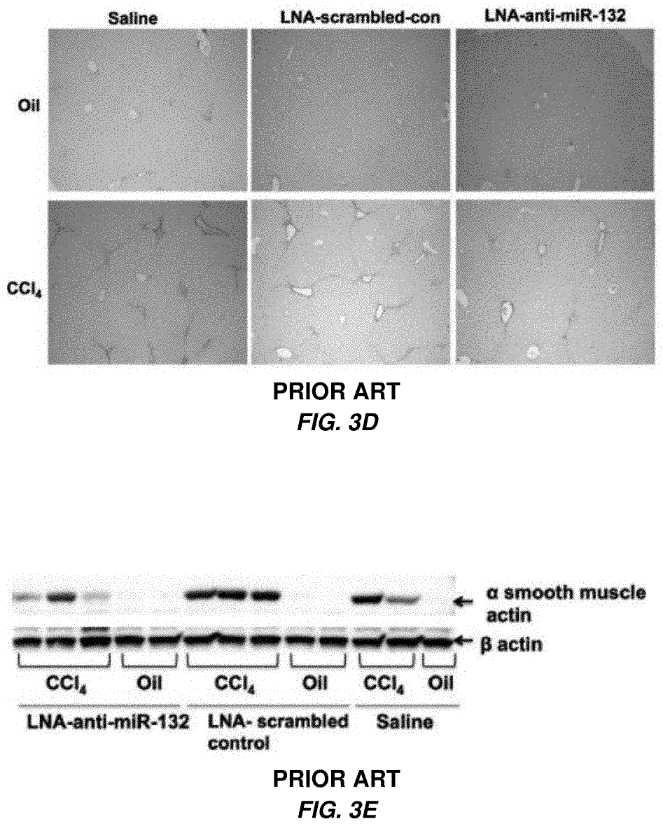

Similar reference characters denote corresponding features consistently throughout the attached drawings.

### DESCRIPTION OF EMBODIMENTS

The compositions for enhancing nucleic acid therapeutics are used in the treatment or prevention of diseases or conditions, such as, for example, cancer or diseases or disorders of the liver. As used herein, “nucleic acid therapeutics” include, but are not limited to, RNA, including small interfering RNA or siRNAs, a small hairpin RNA or shRNAs, microRNA or miRNAs, a double-stranded RNA (dsRNA), an antisense nucleic acid or a locked nucleic acid (LNA), or polynucleotides encoding such with or without chemical modifications. The compositions improve the use of nucleic acid therapeutics with the use of enhancing or stabilizing elements, such as RNA-induced silencing complex (RISC) proteins. The compositions include at least one nucleic acid therapeutic or a polynucleotide encoding at least one nucleic acid therapeutic; at least one enhancing or stabilizing element, mutant, variant or modified form thereof, or a polynucleotide encoding at least one enhancing or stabilizing element, mutant, variant or modified form thereof; and a delivery vehicle, where the delivery vehicle may be exosomes, microvesicles, apoptotic bodies, oncosomes, microparticles, extracellular vesicles, liposomes, nanoparticles, synthetic exosome-inspired vesicles, artificial extracellular vesicle-mimetics, plasmids or vectors. When exosomes or extracellular vesicles are used as the delivery vehicle, the exosomes or the extracellular vesicles may be engineered to be substantially devoid of endogenous nucleic acids by downregulating or inhibiting at least one protein involved in sorting or loading nucleic acids into exosomes or extracellular vesicles. The exosome or the extracellular vesicle may include or may not include at least one targeting moiety or therapeutic molecule expressed on a surface of the exosome or the extracellular vesicle. The making of engineered exosomes or extracellular vesicles substantially devoid of endogenous nucleic acids is described in PCT application no. WO 2021/041473 A1, which is hereby incorporated by reference in its entirety.

As will be discussed in greater detail below, the at least one enhancing or stabilizing element, mutant, variant or modified form thereof, or the polynucleotide encoding at least one enhancing or stabilizing element, mutant, variant or modified form thereof, may be ectopically expressed or overexpressed in the delivery vehicle. As used herein, the term “ectopically expressed” refers to abnormal gene expression, such that the ectopic expression in the delivery vehicle by the at least one enhancing or stabilizing element, mutant, variant or modified form thereof, or the polynucleotide encoding at least one enhancing or stabilizing element, mutant, variant or modified form thereof, is expressed abnormally in the delivery vehicle; i.e., the delivery vehicle, in its normal or unmodified state, would not exhibit this expression. As a non-limiting example, such ectopic expression or overexpression may occur through artificial manipulation of the delivery vehicle.

As a non-limiting example, the vector may be a viral vector. Non-limiting examples of the nucleic acid therapeutic include RNA, at least one small interfering RNA (siRNA), at least one small hairpin RNA (shRNA), at least one microRNA (miRNA), a double-stranded RNA (dsRNA), an antisense nucleic acid, a locked nucleic acid (LNA), a microRNA inhibitor, a chemically-modified microRNA, a chemically-modified siRNA, a chemically-modified shRNA, a chemically-modified dsRNA, a chemically-modified antisense nucleic acid, or a chemically-modified microRNA inhibitor.

Non-limiting examples of the enhancing or stabilizing element include a portion of an RNA-induced silencing complex (RISC) protein, a whole RISC, a mutant, variant or modified form thereof, or any combination of RISC complex. The RISC protein may be Argonaute 2, a mutant, variant or modified form thereof. As another non-limiting example, the enhancing or stabilizing element may be a ribonucleoprotein. The ribonucleoprotein may be GW182, DCP1/CDP2, PUM1, HUR, or mutants, variants or modified forms thereof.

Challenges in the field of cancer therapeutics by RNAi remain because even if RNAis are successfully delivered, changes and dysregulated pathways in transformed cancer cells can downregulate essential factors that affect the silencing efficacy of potential anticancer RNAis, including miR-based therapies and siRNAs. These factors include the RNA-induced silencing complex (RISC) in the recipient cells. RISC is a multiprotein assembly that incorporates one strand of a miR or siRNA and uses them as a template for recognizing and degrading complementary mRNA. If it finds a complementary strand in mRNA, it activates one of the proteins in RISC, called Argonaute 2 (AGO2), activates and cleaves the mRNA. It is well established that hallmarks of cancer, such as hypoxia and genomic loss, are correlated with the downregulation of the RISC complex and can contribute to less activity of anti-cancer miRs in tumors.

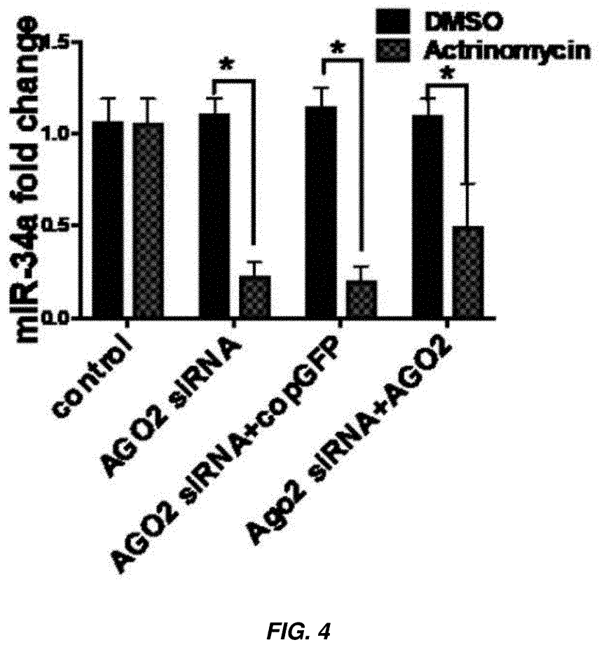

To assess the effect of AGO2, a prototype RISC complex protein, in stabilizing miRs, we silenced AGO2 and halted transcription by using the transcription inhibitor actinomycin D. miR-34a had a low half-life in cells lacking AGO2. Loss of function and rescue experiments showed that AGO2 stabilizes the miR-34a and prolongs its half-life (FIG. 4). AGO2 was successfully delivered to the cells showing that stabilizing elements can be delivered via transfection reagents and liposomes to increase the stability of miRNA and other nucleic acid therapeutics. This work demonstrated the potential benefit of co-delivering a RISC complex protein such as AGO2 together with nucleic acid therapeutics to increase their stability and efficacy. FIG. 4 is a graph illustrating the silencing of AGO2 in an oral cancer cell line, both with or without copGFP plasmid or AGO2 overexpression plasmid. Actinomycin D was administered, and levels of miR-34a were determined by RT-qPCR. The data in FIG. 4 are presented as mean/standard deviation (SD) and are representative of three independent experiments. In FIG. 4, the * indicates p<0.05. Other RNA binding proteins that can stabilize or destabilize RNA have been reported, including HuR/HuA, HuB, HuC, HuD, AUF1, GW182, DCP1/CDP2, PUM1, Tristetraprolin (TTP), and KSRP. Using a similar approach, they can be targeted or delivered to increase the stability of therapeutic RNA in the recipient cells.

Mutants, variants, and modified forms of the enhancing or stabilizing elements can also be used in the present compositions and methods. For example, the AGO2 Y529F mutation will prevent phosphorylation, which is a cellular mechanism to decrease AGO2 activity. Delivery of AGO2 Y529F will prevent this inactivation. Other modified forms of AGO2 include P700 prolyl 4-hydroxylation, S253, T303, T307, S798 phosphorylation, S387 phosphorylation, Y393 phosphorylation, ubiquitination and PARylation.

The delivery of the RISC complex protein or other stabilizing factor together with the nucleic acid therapeutic(s) can be accomplished using many different delivery vehicles, including, but not limited to, a synthetic or natural delivery vehicle, such as exosomes, microvesicles, apoptotic bodies, oncosomes, extracellular vesicles, microparticles, liposomes or nanoparticles. It can also be accomplished by delivering genetic material encoding for RISC genes together with nucleic acid-based therapeutic(s) using plasmids and vectors.

Extracellular vesicles are membrane enclosed vesicles released by cells. Their primary constituents are lipids, proteins and nucleic acids. They are composed of a lipid-protein bilayer encapsulating an aqueous core comprising nucleic acids and soluble proteins. Extracellular vesicles include, but are not limited to, exosomes, shedding vesicles, microvesicles, small vesicles, large vesicles, microparticles, and apoptotic bodies, based on their size, cellular origin and formation mechanism. Exosomes are formed by inward budding of late endosomes forming multivesicular bodies (MVB), which then fuse with the limiting membrane of the cell concomitantly releasing the exosomes. Shedding vesicles are formed by outward budding of the limiting cell membrane followed by fusion. When a cell undergoes apoptosis, the cell disintegrates and divides its cellular content in different membrane enclosed vesicles, referred to as “apoptotic bodies.” Non-limiting examples of extracellular vesicles include circulating extracellular vesicles, beta cell extracellular vesicles, islet cell extracellular vesicles, exosomes and apoptotic bodies, and combinations thereof.

Large extracellular vesicles can range from about 5 μm to about 12 μm in diameter. Apoptotic bodies can range from about 1 μm to about 5 μm in diameter. Microvesicles can range from about 100 nm to about 1 μm in diameter. Exosomes can range from about 30 nm to about 150 nm, from about 30 nm to about 100 nm, or from about 50 nm to about 150 nm in diameter or from about 50 nm to about 200 nm.

Extracellular vesicles or exosomes may be isolated or derived from bone marrow, red blood cells, tumor cells, immune cells, epithelial cells, fibroblasts, or stem cells. Extracellular vesicles or exosomes may be isolated or derived from B cells, T cells, monocytes, or macrophages. In one embodiment, extracellular vesicles or exosomes to be taken up by a specific type of cells (e.g., monocytes/macrophages) are isolated or derived from the same type of cells (e.g., monocytes/macrophages).

Extracellular vesicles or exosomes may be isolated or derived from a body fluid. For example, the body fluids may include, but are not limited to, serum, plasma, blood, whole blood and derivatives thereof, urine, tears, saliva, sweat, cerebrospinal fluid (CSF), oral mucus, vaginal mucus, seminal plasma, semen, prostatic fluid, excreta, ascites, lymph, bile, breast milk and amniotic fluid.

Additionally, extracellular vesicles or exosomes may be isolated or derived from cultured cells.

Methods for isolating extracellular vesicles include size separation methods, such as centrifugation. In one embodiment, isolating various components of extracellular vesicles may be performed through an isolation method including sequential centrifugation. The method may include centrifuging a sample at 800 g for a desired amount of time, collecting the pellet containing cells and cellular debris and (first) supernatant, centrifuging the (first) supernatant at 2,000 g for a desired time, and collecting the pellet containing large extracellular vesicles and apoptotic bodies and (second) supernatant. The sequential centrifugation method can further include centrifuging the (second) supernatant at 10,000 g, and collecting the pellet containing microvesicles and (third) supernatant. The sequential centrifugation method can further include centrifuging the (third) supernatant at 100,000 g, and collecting the pellet containing exosomes (ranging from about 30 nm to about 200 nm in diameter) and (fourth) supernatant.

The sequential centrifugation method can further include washing each of the pellets including the extracellular vesicles (e.g., large extracellular vesicles and apoptotic bodies, microvesicles, and exosomes), such as in phosphate buffered saline, followed by centrifugation at the appropriate gravitational force and collecting the pellet containing the extracellular vesicles. Isolation, purity, concentration, size, size distribution, and combinations thereof of the extracellular vesicles following each centrifugation step can be confirmed using methods such as nanoparticle tracking, transmission electron microscopy, immunoblotting, and combinations thereof. Nanoparticle tracking (NTA) to analyze extracellular vesicles, such as for concentration and size, can be performed by dynamic light scattering using commercially available instruments such as certain ZetaView® brand instruments (commercially available from Particle Metrix of Meerbusch, Germany). Following isolation, the method can further include detecting an extracellular vesicle marker.

Methods for isolating extracellular vesicles also include using commercially available reagents such as, for example, the ExoQuick-TC™ brand reagent (commercially available from System Biosciences of Palo Alto, California).

Exosomes are small vesicular bodies that are secreted from cells into the cellular microenvironment and biofluids and can enter both neighboring cells and the systemic circulation. Exosomes are actively assembled from intracellular multivesicular bodies (MVBs) by the endosomal sorting complex required for transport (ESCRT) machinery. Exosomes contain various molecular constituents of their cell of origin, including, but not limited to, proteins, RNA (such as mRNA, miRNA, etc.), lipids and DNA.

Exosomes may be isolated by any suitable techniques, including ultracentrifugation, micro-filtration, size-exclusion chromatography, etc. or a combination thereof. Exosomes can be isolated using a combination of techniques based on both physical (e.g., size and density) and biochemical parameters (e.g., presence/absence of certain proteins involved in their biogenesis). In certain embodiments, exosomes are isolated using a kit. In one embodiment, exosomes are isolated using the Total Exosome Isolation Kit and/or the Total Exosome Isolation Reagent from Invitrogen®.

Following isolation, the method can further include detecting an extracellular vesicle marker of the extracellular vesicle. Extracellular vesicle or exosome markers include CD9, CD63, CD81, LAPM1, TSG101, and combinations thereof. Exosomes are nanosized (50-150 nm) membrane bounded vesicles secreted by almost all types of cells and are found in biofluids. They naturally carry biomacromolecules, including different RNAs (mRNAs, miRs), DNA, lipids, and proteins, and can efficiently deliver their cargoes to recipient cells, eliciting functions and mediating cellular communications. Previous work has shown the advantages of using exosomes/extracellular vesicles for drug delivery, including: 1) exosomes are small and have a high efficiency for delivery due to their similarity to cell membranes; 2) exosomes are biocompatible, non-immunogenic, and non-toxic, even in repeated in vivo injections; 3) exosomes are stable even after several freeze and thaw cycles, and their lipid bilayer protects the protein and RNA cargoes from enzymes such as proteases and RNases; 4) exosomes have a slightly negative zeta potential, leading to long circulation; and 5) exosomes also exhibit an increased capacity to escape degradation or clearance by the immune system.

Previous experiments have shown that exosomes can functionally deliver miR inhibitors, RNAis, and miRs. Exosomes were able to successfully deliver miR-155 analogue to the liver and hepatocytes of miR-155 KO mice, and can restore the level to up to 50% of wild type mice. We have optimized the protocol of loading exosomes with miRs, miR inhibitors, and siRNAs. Repeated injection of exosomes did not induce any adverse effects, cytotoxicity, and immunogenicity in vivo and in vitro.

One challenge with using exosomes for delivery of RNAi, RISC complex proteins or nucleic acid stabilizing proteins is that they contain endogenous RNA that might be harmful to the patient, such as by promoting tumorigenesis, exacerbating diseases such as cancer. Thus, delivering RISC complex proteins together with potentially harmful endogenous RNA may intensify adverse events. We therefore produced our engineered safeEXO (ES-exo) platform that manipulates the exosome creation machinery in producer cells to significantly minimize endogenous RNA loading into exosomes.

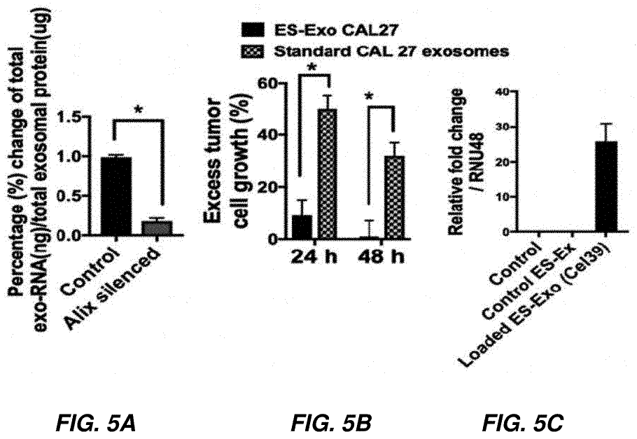

To produce ES-exo, we have silenced ALIX in CAL27 and found a significant decrease in RNA content of exosomes (FIG. 5A). Functionally, these exosomes did not induce cell proliferation after 24 hours and 36 hours coculture with recipient CAL27 cells, indicating that removal of their endogenous nucleic acids makes them safe and non-tumorigenic (FIG. 5B). ES-exo were loaded with synthetic Caenorhabditis elegans miR-39 (Cel-39 is not present in human) and were able to deliver Cel-39 to the recipient CAL27 cells in 4 hours (FIG. 5C). Thus, the production of our ES-exo platform should prevent harmful effects of endogenous exosome RNA when they are loaded with RISC complex proteins and nucleic acid therapeutic(s), such as AGO2 and mutant AGO2, such as AGO2 Y529F mutation or any other mutations that can modulate its activity. FIG. 5A is a graph comparing CAL27 cells treated with Alix siRNA against a control, where the media was changed after 16 hours and the exosomes were isolated after 48 hours. Levels of exosomal RNA content were normalized to exosomal protein (n=4 per group). FIG. 5B is a graph comparing CAL27 cells treated with standard CAL27 exosomes and engineered exosomes derived from CAL27. Cell growth was quantified in tumor cells by MTT assay (n=at least 3 in each condition). FIG. 5C is a graph comparing engineered exosomes loaded with Ce139 against controls, where the level of the miR was quantified in the recipient cells by RT-qPCR (n=4 per group). In FIGS. 5A and 5B, the * indicates statistically significant data.

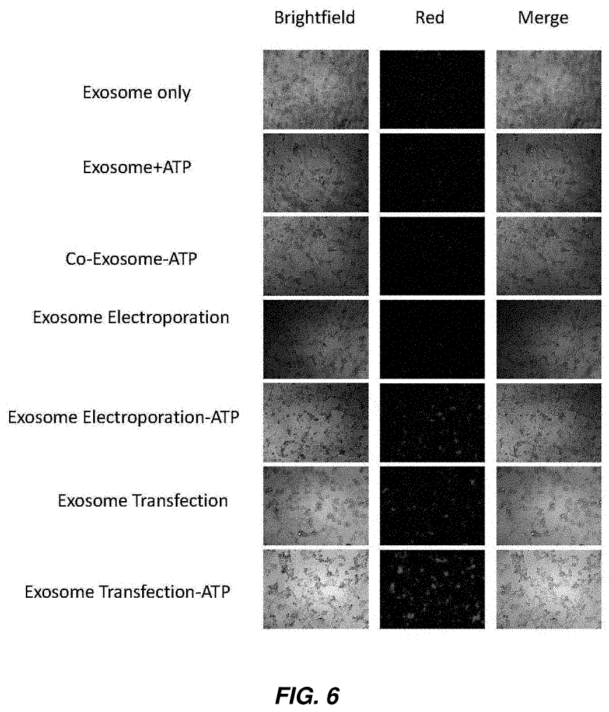

ES-exo was transfected with an exosome transfection reagent from the Exo-Fect™ siRNA/miRNA Transfection Kit, manufactured by System Biosciences®, following the manufacturer's protocol, or was electroporated with ATP, or co-incubated with 10 mM ATP. Further, ES-exos were labeled PKH26 red fluorescent dye (Sigma® #MINI26-1KT) according to the manufacturer's protocol and were further filtered through a 100 kda filter to remove any further suspended dye. KRAS 4B wild type cells were then seeded in 48 wells (40,000 cells per well) and were treated with PKH26 red fluorescent labeled ATP transfected or untransfected ES-exo. After 2 hours, the wells were washed twice with 1X PBS and were imaged using a ZOE™ Fluorescent Cell Imager, manufactured by Bio-Rad® Laboratories. The images are shown in FIG. 6.

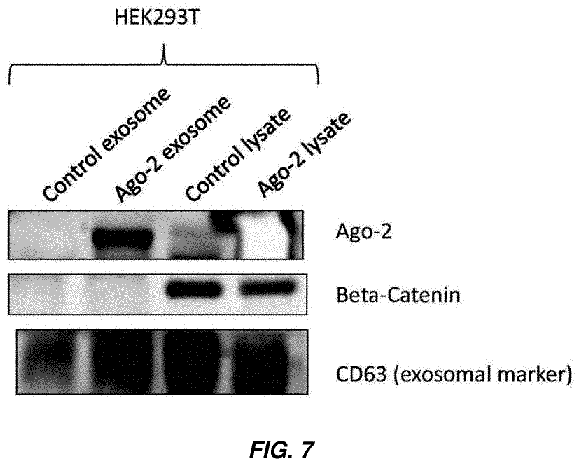

Further, HEK293T cells were transfected with an Ago2 expressing plasmid. The cells were collected and lysed, and exosomes were isolated from the culture media. The exosomes were isolated using filtration and Exoquick™ from System Biosciences®. 100 μg of protein from the cell and exosome lysate were used for a western blot. CD63 was used as the marker for exosomes. Beta catenin was used as the positive control for the cell lysates. As shown in FIG. 7, the exosomes derived from Ago2 transfected cells showed an increase in expression and packaging of Ago2 in the exosomes.

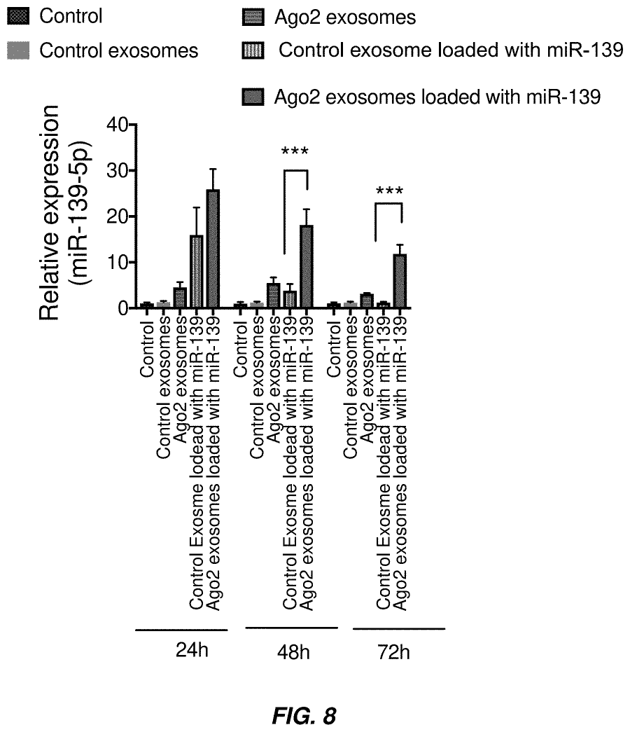

Additionally, 25 μg of control exosomes, Ago2 expressing exosomes, control exosomes loaded with miR-139-5p, and Ago2 expressing exosomes loaded with miR-139-5p were co-cultured with HEK293T cells. The cells were lysed and RNA was isolated after 24 hours, 48 hours, and 72 hours, followed by qRT-PCR. RNU48 was used as a normalizer. The results are shown in FIG. 8, where the results are presented as fold change compared to the control, based on the delta-delta ct method. As shown in FIG. 8, the results indicate stabilization of RNA at 48 hours and 72 hours by the Ago2 expressing exosomes loaded with miR-139-5p. In FIG. 8, *** indicates p<0.0001.

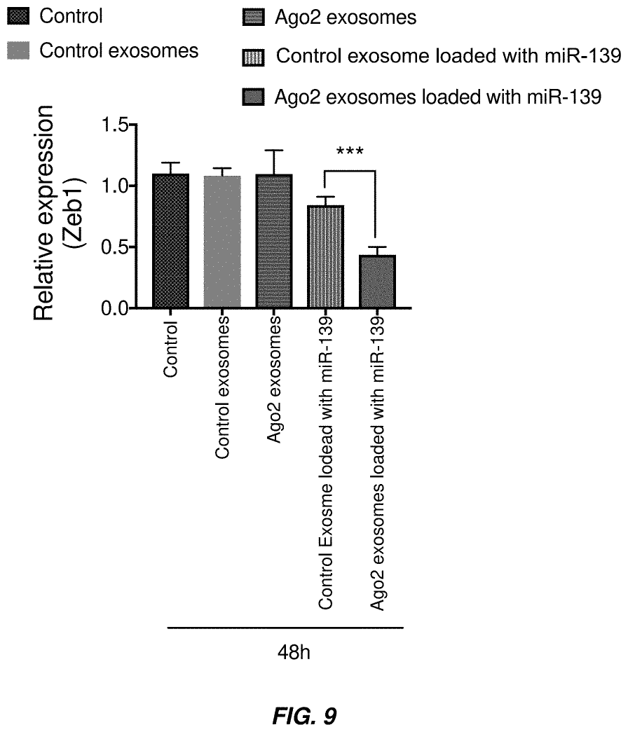

As shown in FIG. 9, 48 hours after the experimental conditions indicated above for FIG. 8, RNA was isolated from the cells and qRT-PCR for ZEB1 was performed. ZEB1 is the direct target of miR-139-5p and its inhibition indicates functionality of miR-139-5p delivered by exosomes. As can be seen in FIG. 9, Ago2 exosomes loaded with miR-139-5p significantly induced suppression of ZEB1 mRNA. The results in FIG. 9 are presented as fold change compared to the control based on the delta-delta ct method. In FIG. 9, *** indicates p<0.0001.

The formation and use of liposomes are generally known to those having ordinary skill in the art. Recently, liposomes were developed with improved serum stability and circulation half-times, as discussed in U.S. Pat. No. 5,741,516, which is hereby incorporated by reference in its entirety. Further, various methods of liposome and liposome-like preparations as potential drug carriers have been described, such as discussed in U.S. Pat. Nos. 5,567,434; 5,552,157; 5,565,213; 5,738,868; and 5,795,587, each of which is hereby incorporated by reference in its entirety.

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also referred to as “multilamellar vesicles” or MLVs). MLVs generally have diameters of from 25 nm to 4 μm. Sonication of MLVs results in the formation of small unilamellar vesicles (SUVs) with diameters in the range of 200 Å to 500 Å, containing an aqueous solution in the core.

Alternatively, nanocapsule formulations may be used. Nanocapsules can generally entrap substances in a stable and reproducible way. Nanoparticles are a colloidal carrier system that has been shown to improve the efficacy of an encapsulated drug by prolonging the serum half-life. Polyalkylcyanoacrylates (PACAs) nanoparticles are a polymer colloidal drug delivery system that is in clinical development. Biodegradable poly (hydroxyl acids), such as the copolymers of poly (lactic acid) (PLA) and poly (lactic-co-glycolide) (PLGA) are being extensively used in biomedical applications and have received FDA approval for certain clinical applications. In addition, nanoparticles have many desirable carrier features, including: 1) that the agent to be encapsulated comprises a reasonably high weight fraction (loading) of the total carrier system; 2) that the amount of agent used in the first step of the encapsulation process is incorporated into the final carrier (entrapment efficiency) at a reasonably high level; 3) that the carrier has the ability to be freeze-dried and reconstituted in solution without aggregation; 4) that the carrier be biodegradable; 5) that the carrier system be of small size; and 6) that the carrier enhances the particles persistence.

Nanoparticles may be synthesized using virtually any biodegradable shell known in the art. Such polymers are biocompatible and biodegradable and are subject to modifications that desirably increase the photochemical efficacy and circulation lifetime of the nanoparticle. In one embodiment, the polymer is modified with a terminal carboxylic acid group (COOH) that increases the negative charge of the particle and thus limits the interaction with negatively charged nucleic acids. Nanoparticles may also be modified with polyethylene glycol (PEG), which also increases the half-life and stability of the particles in circulation. Alternatively, the COOH group may be converted to an N-hydroxysuccinimide (NHS) ester for covalent conjugation to amine-modified compounds.

As used herein, a “vector” may be any of a number of nucleic acids into which a desired sequence or sequences may be inserted for transport between different genetic environments or for expression in a host cell. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. Vectors include, but are not limited to, viral vectors, plasmids, cosmids, fosmids, phages, phage lambda, phagemids, and artificial chromosomes.

Vectors typically include the DNA of a transmissible agent, into which foreign DNA is inserted. A common way to insert one segment of DNA into another segment of DNA involves the use of enzymes called restriction enzymes, which cleave DNA at specific sites (i.e., specific groups of nucleotides) called restriction sites. A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.”

A common type of vector is a “plasmid”, which generally is a self-contained molecule of double-stranded DNA, usually of bacterial origin, that can readily accept additional (foreign) DNA and which can readily be introduced into a suitable host cell. A plasmid vector often contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA may be from the same gene or from different genes and may be from the same or different organisms.

A large number of vectors, including plasmid and fungal vectors, have been described for replication and/or expression in a variety of eukaryotic and prokaryotic hosts, and many appropriate host cells, are known to those having ordinary skill in the art. Recombinant cloning vectors will often include one or more replication systems for cloning or expression, one or more markers for selection in the host (e.g. antibiotic resistance), and one or more expression cassettes.

Viral vectors may be derived from DNA viruses or RNA viruses, which have either episomal or integrated genomes after delivery to the cell. Viral vectors may be derived from retroviruses (including lentiviruses), replication defective retroviruses (including replication defective lentiviruses), adenoviruses, replication defective adenoviruses, adeno-associated viruses (AAV), herpes simplex viruses, and poxviruses. In some embodiments, the vector is a lentiviral vector. Options for gene delivery of viral constructs are well known in the art.

Any subtype, serotype and pseudotype of lentiviruses, and both naturally occurring and recombinant forms, may be used as a vector for the present compositions and methods. Lentiviral vectors may include, without limitation, primate lentiviruses, goat lentiviruses, sheep lentiviruses, horse lentiviruses, cat lentiviruses, and cattle lentiviruses.

The term AAV covers all subtypes, serotypes and pseudotypes, and both naturally occurring and recombinant forms. AAV viral vectors may be selected from among any AAV serotype, including, without limitation, AAV1, AAV2, AAV3, AAV4, AAV5, AAV6, AAV7, AAV8, AAV9, AAV10 or other known and unknown AAV serotypes. Pseudotyped AAV refers to an AAV that contains capsid proteins from one serotype and a viral genome of a second serotype.

With regard to treatment using the present compositions, the conditions to be treated include, but are not limited to, cancer, including, but not limited to, hematologic malignancy, lung cancer, ear, nose and throat cancer, colon cancer, melanoma, pancreatic cancer, mammary cancer, prostate cancer, breast cancer, ovarian cancer, basal cell carcinoma, biliary tract cancer, bladder cancer, bone cancer, breast cancer, cervical cancer, choriocarcinoma, colon and rectum cancer, connective tissue cancer, cancer of the digestive system, endometrial cancer, esophageal cancer, eye cancer, cancer of the head and neck, gastric cancer, intra-epithelial neoplasm, kidney cancer, larynx cancer, liver cancer, fibroma, neuroblastoma, oral cavity cancer (e.g., lip, tongue, mouth, and pharynx), ovarian cancer, pancreatic cancer, prostate cancer, retinoblastoma, rhabdomyosarcoma, rectal cancer, renal cancer, cancer of the respiratory system, sarcoma, skin cancer, stomach cancer, testicular cancer, thyroid cancer, uterine cancer, cancer of the urinary system, as well as other carcinomas and sarcomas. The present compositions may also be used to treat liver disease, including, but not limited to, Alcoholic Liver Disease (ALC), hepatocellular carcinoma (HCC), non-alcoholic steatohepatitis (NASH), hepatitis C viral infection (HCV), non-alcoholic fatty liver disease (NAFLD), and liver fibrosis.

It is to be understood that the compositions and methods for enhancing nucleic acid therapeutics is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A therapeutic composition, comprising:
at least one nucleic acid therapeutic or a polynucleotide encoding at least one nucleic acid therapeutic;
at least one enhancing or stabilizing element, mutant, variant or modified form thereof, or a polynucleotide encoding at least one enhancing or stabilizing element, mutant, variant or modified form thereof; and
a delivery vehicle, wherein the delivery vehicle is selected from the group consisting of exosomes, microvesicles, apoptotic bodies, oncosomes, microparticles, extracellular vesicles, liposomes, nanoparticles, synthetic exosome-inspired vesicles, artificial extracellular vesicle-mimetics, plasmids, and vectors.

2. The therapeutic composition as recited in claim 1, wherein the exosomes or the extracellular vesicles are engineered to be substantially devoid of endogenous nucleic acids by downregulating or inhibiting at least one protein involved in sorting or loading nucleic acids into exosomes or extracellular vesicles.

3. The therapeutic composition as recited in claim 1, wherein each of the exosomes or the extracellular vesicles comprises at least one targeting moiety or therapeutic molecule expressed on a surface of the respective exosome or extracellular vesicle.

4. The therapeutic composition as recited in claim 1, wherein the vector comprises a viral vector.

5. The therapeutic composition as recited in claim 1, wherein the nucleic acid therapeutic comprises RNA.

6. The therapeutic composition as recited in claim 1, wherein the nucleic acid therapeutic is selected from the group consisting of at least one small interfering RNA (siRNA), at least one small hairpin RNA (shRNA), at least one microRNA (miRNA), a double-stranded RNA (dsRNA), an antisense nucleic acid, a chemically-modified miRNA, a chemically-modified siRNA, a chemically-modified shRNA, a chemically-modified dsRNA, a chemically-modified antisense nucleic acid, a locked nucleic acid (LNA), and combinations thereof.

7. The therapeutic composition as recited in claim 1, wherein the nucleic acid therapeutic is selected from the group consisting of microRNA, microRNA inhibitor, chemically-modified microRNA, and chemically-modified microRNA inhibitor.

8. The therapeutic composition as recited in claim 1, wherein the enhancing or stabilizing element is a portion of an RNA-induced silencing complex (RISC) protein, a whole RISC, a mutant, variant or modified form thereof, or any combination of RISC complex.

9. The therapeutic composition as recited in claim 8, wherein the RISC protein is Argonaute 2, a mutant, variant or modified form thereof.

10. The therapeutic composition as recited in claim 1, wherein the enhancing or stabilizing element is a ribonucleoprotein.

11. The therapeutic composition as recited in claim 10, wherein the ribonucleoprotein is selected from the group consisting of GW182, DCP1/CDP2, PUM1, HUR, and mutants, variants and modified forms thereof.

12. The therapeutic composition as recited in claim 1, wherein the at least one enhancing or stabilizing element, mutant, variant or modified form thereof, or the polynucleotide encoding at least one enhancing or stabilizing element, mutant, variant or modified form thereof, is ectopically expressed or overexpressed in the delivery vehicle.

13. A method of treating or preventing a disease or disorder, comprising the step of administering to a patient in need thereof a therapeutically effective amount of a therapeutic composition, wherein the therapeutic composition comprises:
at least one nucleic acid therapeutic or a polynucleotide encoding at least one nucleic acid therapeutic;
at least one enhancing or stabilizing element, mutant, variant or modified form thereof, or a polynucleotide encoding at least one enhancing or stabilizing element, mutant, variant or modified form thereof; and
a delivery vehicle, wherein the delivery vehicle is selected from the group consisting of exosomes, microvesicles, apoptotic bodies, oncosomes, microparticles, extracellular vesicles, liposomes, nanoparticles, plasmids and vectors.

14. The method of treating or preventing a disease or disorder as recited in claim 13, wherein the exosomes or the extracellular vesicles are engineered to be substantially devoid of endogenous nucleic acids by downregulating or inhibiting at least one protein involved in sorting or loading nucleic acids into exosomes or extracellular vesicles.

15. The method of treating or preventing a disease or disorder as recited in claim 14, wherein each of the exosomes or the extracellular vesicles comprises at least one targeting moiety or therapeutic molecule expressed on a surface of the respective exosome or extracellular vesicle.

16. The method of treating or preventing a disease or disorder as recited in claim 13, wherein the vector comprises a viral vector.

17. The method of treating or preventing a disease or disorder as recited in claim 13, wherein the nucleic acid therapeutic is selected from the group consisting of at least one small interfering RNA (siRNA), at least one small hairpin RNA (shRNA), at least one microRNA (miRNA), a double-stranded RNA (dsRNA), an antisense nucleic acid, a locked nucleic acid (LNA), and combinations thereof.

18. The method of treating or preventing a disease or disorder as recited in claim 13, wherein the enhancing or stabilizing element is a portion of an RNA-induced silencing complex (RISC) protein, a whole RISC, a mutant, variant or modified form thereof, or any combination of RISC complex.

19. The method of treating or preventing a disease or disorder as recited in claim 13, wherein the enhancing or stabilizing element is a ribonucleoprotein selected from the group consisting of GW182, DCP1/CDP2, PUM1, HUR, and mutants, variants and modified forms thereof.

20. The method of treating or preventing a disease or disorder as recited in claim 13, wherein the at least one enhancing or stabilizing element, mutant, variant or modified form thereof, or the polynucleotide encoding at least one enhancing or stabilizing element, mutant, variant or modified form thereof, is ectopically expressed or overexpressed in the delivery vehicle.

21. A delivery vector that delivers the following:
(a) at least one RISC complex protein or fragment; or
(b) nucleic acid therapeutics with nucleic acid that encodes for at least one RISC complex protein or fragment; or
(c) a combination thereof.
